(19) United States
(12) Patent Application Publication
(10) Pub. No.: US 2025/0325612 A1
(43) Pub. Date: Oct. 23, 2025

(54) Method for Extraction and Preparation of Anti-Inflammatory Ovary-Protecting Herb

(71) Applicant: Andall Biosciences Inc., Torrance, CA (US)

(72) Inventor: Heju Li, Torrance, CA (US)

(21) Appl. No.: 18/735,849

(22) Filed: Jun. 6, 2024

(30) Foreign Application Priority Data
Apr. 17, 2024 (CN) 202410462990.4

(51) Int. Cl. A61K 36/282; A61K 9/20; A61K 36/234; A61K 36/284; A61K 36/489; A61K 36/756

(52) U.S. Cl. CPC A61K 36/282; A61K 9/2095; A61K 36/234; A61K 36/284; A61K 36/489; A61K 36/756; A61K 2236/331; A61K 2236/333

(57) ABSTRACT

The present disclosure relates to the technical field of herbal extraction and preparation, and particularly to a method for extraction and preparation of an anti-inflammatory ovary-protecting herb. The method comprises an internal drug and an external drug, wherein the external drug comprises 86 g of Sophora flavescens, 182 g of Fructus cnidii, 66 g of Phellodendron amurense, 146 g of Rhizoma atractylodis, 76 g of Capillary artemisia, matrix S-40, sodium dihydrogen phosphate and polysorbitol-80. The Sophora flavescens and Fructus cnidii in the external drug of the present disclosure have the functions of clearing heat and dampness, dispelling wind, killing insects and relieving itching. Through scientific extraction, the Sophora flavescens and Fructus cnidii combined with adjuvant medicinal materials such as Phellodendron amurense, Rhizoma atractylodis and Capillary artemisia are prepared into an effervescent suppository.

### CROSS REFERENCE TO RELATED APPLICATION

This application claims the benefit of and takes priority from Chinese Patent Application No. 202410462990.4 filed on Apr. 17, 2024, the contents of which are herein incorporated by reference.

### TECHNICAL FIELD

The present disclosure relates to the technical field of extraction and preparation of herbs, and particularly to a method for extraction and preparation of an anti-inflammatory ovary-protecting herb.

### BACKGROUND

Gynecological inflammation is reproductive system infection caused by damage of protective tissues due to invasion of bacteria, viruses, fungi and the like, including common vulvitis, vaginitis and cervicitis, as well as endometritis, salpingitis, salpingo oophoritis, pelvic peritonitis and pelvic connective tissue inflammation, etc.

When women's ovarian suffers from diseases, it can cause reduction in female estrogen levels. The reduction in female estrogen levels can make women have unclear secondary sexual characteristics, skin pigmentation, rough skin and the low immunity of a female reproductive system, menstrual disorder (rare) and causes a pain in the back, and even can affect normal ovulation to lead to infertility and other problems. The too low estrogen level can cause a relatively high androgen level, thereby leading to facial acne, male type hair loss (Mediterranean alopecia, and alopecia areata), and in severe cases, it can cause polycystic ovary syndrome (PCOS). Furthermore, when women have ovarian diseases, it is necessary to radically treat the diseases, at this moment, the inflammatory of ovaries needs to be diminished to avoid the risk of secondary infection. Moreover, after the radical treatment of ovarian diseases, the estrogen level drops significantly, at this moment, we need to supplement estrogen. The existing estrogen supplements can cause adverse symptoms in people, however, women have to take this type of drugs to help recovery, women need to take drugs containing estrogen during their maintenance, which can help maintain the ovaries, but excessive estrogen level can lead to inflammation, and therefore two complementary drugs are required. Therefore, we propose a method for extraction and preparation method of an anti-inflammatory ovary-protecting herb in order to solve the above problems.

### SUMMARY

The objective of the present disclosure is to provide a method for extraction and preparation of an anti-inflammatory ovary-protecting herb in order to solve the problems proposed in the above background.

The technical solution of the present disclosure is a method for extraction and preparation of an anti-inflammatory ovary-protecting herb, comprising an internal drug and an external drug;
wherein the external drug comprises 86 g of Sophora flavescens, 182 g of Fructus cnidii, 66 g of Phellodendron amurense, 146 g of Rhizoma atractylodis, 76 g of Capillary artemisia, matrix S-40, sodium dihydrogen phosphate, and polysorbitol-80;
a decoction method is used to prepare the external drug, which comprises the following steps:

Step S1: weighing 86 g of Sophora flavescens, 182 g of Fructus cnidii, 66 g of Phellodendron amurense, 146 g of Rhizoma atractylodis and 76 g of Capillary artemisia, washing the above five medicine herbs and then placing the medicinal materials in a decoction pot, adding water above the surfaces of the medicinal materials to soak the medicinal materials for 30 min, decocting twice in total based on the decoction method, and then filtering using a filter screen to obtain filtrate; wherein the treatment and decoction method of the medicinal materials is as follows:
(1) removing impurities in Sophora flavescens, fructus cnidii, Phellodendron amurense, Rhizoma atractylodis and Capillary artemisia, removing residual stems, soaking the above medicinal materials with water, fishing out the above medicinal materials, moisturizing, slicing, and drying for later use;
(2) cleaning up all the medicinal materials and then pouring the cleaned medicinal materials into a medicinal material tank, followed by pouring water into the medicinal material tank for decoction;
(3) putting 10 times the amount of water for the first decoction so that water covers the medicinal materials, and then setting decoction time as an hour and a half,
(4) collecting the liquid medicine herb obtained after the first decoction, putting 8 times the amount of water for the second decoction, and then setting decoction time as an hour and a half; and
(5) filtering two liquid medicinal materials, and then respectively placing two filtrates in different vessels;

Step S2: merging the two filtrates, performing vacuum concentration to 1000 ml, adding 3 times the amount of 95% ethanol after cooling while stirring so that the content of alcohol in the merged filtrate reaches about 70%, placing for 24 h, filtering, collecting filtrate, recovering ethanol from the filtrate to form a solution, and then evaporating the solution until an extract is formed for later use; wherein the method for preparing the solution into the extract is as follows:
(1) shaking the collected solution, then putting the solution into a beaker and heating the solution, wherein in the process of heating, the temperature is raised so that ethanol is evaporated out; and
(2) adjusting the temperature to soft fire so that the solution is decocted to form the extract, and the density of the extract is 1.41-1.44 g/ene;

Step S3: heating and melting the matrix S-40 in a water bath, adding the above extract, sodium dihydrogen phosphate and polysorbitol ester-80 under the condition of stirring, evenly mixing the above materials, pouring the obtained mixture into a bolt mould, cooling, and demoulding to obtain an ointment; wherein the preparation and formation method of the ointment is as follows:
(1) putting the extract, then evenly mixing sodium dihydrogen phosphate and polysorbitol ester-80, then pouring the obtained mixture into the extract, and subsequently adjusting the fire power to soft fire in the process of stirring;
(2) observing the viscosity of the ointment in the process of stirring until the ointment juice is not broken after being pulled, or the ointment juice is dropped into clean water to form beads without dispersing; and
(3) putting the cooled ointment into a clean mould, covering the mouth of the mould with clean gauze before being not covered with a lid, placing the ointment overnight until complete cooling, and then taking out the ointment, that is, obtaining the ointment;
the internal drug comprises estradiol, sodium dodecyl sulfate, cross-linked polyvinylpyrrolidone, polyvinylpyrrolidone, cetanol, tartaric acid, potassium hydrogen tartrate, boric acid, sodium bicarbonate, magnesium stearate and sodium carboxymethyl cellulose;
the specific steps for preparing tablets using an acid and alkali separated granulation method are as follows:

Step S4: screening estradiol via a 120-mesh sieve, and screening sodium carboxymethyl cellulose via a 80-mesh sieve for later use; wherein the method for grinding into powders is as follows:
(1) taking out the estradiol and removing a sugar coating outside the estradiol, and then grinding the estradiol into powders, wherein during the grinding, the ground powders are required to pass through a sieve with a size of 0.125 mm; and
(2) grinding the sodium carboxymethyl cellulose into powders capable of passing through a sieve with a size of 0.180 mm, and respectively putting the above two drugs into different vessels;

Step S5: evenly mixing the estradiol, boric acid, potassium hydrogen tartrate and tartaric acid, then adding the sodium carboxymethyl cellulose, 10% polyvinylpyrrolidone, and an anhydrous ethanol solution into the above mixture to prepare a soft material, and granulating after passing through a 20-mesh sieve; and drying wet particles in a thermostatic blower to obtain particles A; wherein the drying method of the particles A is as follows:
(1) mixing several materials and then adding the 10% polyvinylpyrrolidone and anhydrous ethanol into the above mixture;
(2) then twisting the obtained mixture into the particles A capable of passing through holes with a size of 1.27 mm during the stirring; and
(3) placing the twisted particles A in a drier, wherein each twisted particle is placed properly, and a distance between every two particles is equal;

Step S6: evenly mixing sodium bicarbonate, cross-linked polyvinylpyrrolidone, sodium dodecyl sulfate and cetanol, and then granulating using the same method, drying, and granulating; wherein the obtained particles are particles B, evenly mixing the two particles, then adding magnesium stearate into the above mixture to be mixed evenly, then measuring the content of the estradiol followed by determining the weight of the tablet, and tabletting; wherein the method for preparing the particles AB into the tablet is as follows:
(1) mixing and stirring the sodium bicarbonate, cross-linked polyvinylpyrrolidone, sodium dodecyl sulfate and cetanol, and observing the stirring;
(2) adding the 10% polyvinylpyrrolidone and anhydrous ethanol into the stirred liquid to be mixed, then twisting the particles B into small particles capable of passing through a sieve with a size of 1.27 mm, and then drying the particles B; and
(3) stirring the dried particles AB while putting magnesium stearate, then weighing the mixed particles AB on an electronic scale, and preparing the mixed particles into complete particles using a stamping tablet press when the weight of the mixed particles AB meets the standard.

Preferably, in the step S1, the filter screen during the filtration is 0.150 mm, and the material of the filter screen is a nylon material.

Preferably, in the step S2, an instrument for vacuum concentration is a rotary evaporator, and the vacuum degree in the instrument is 560 mmHg.

Preferably, in the step S2, the recovery of ethanol from the filtrate adopts a distillation method, and the heating temperature of the filtrate is 78-90° C.

Preferably, in the step S3, the mould is cylindrical, and the height of the mould is 0.2 m.

Preferably, in the step S4, an instrument for grinding is a mortar, and the material of the mortar is porcelain.

Preferably, in the step S5, the time of wet mixing and stirring is 2-3 min, and the drying temperature is 35-45° C.

Preferably, in the step S5, a device for twisting into particles is an oscillating granulator, and the material of the sieve is stainless steel.

Preferably, in the step S6, the model of the stamping tablet press is a dual-discharge tablet press, and the weight of the tablet is 1 g.

Through improvement, the present disclosure provides a method for extraction and preparation of an anti-inflammatory ovary-protecting herb here. Compared with the prior art, the present disclosure has the following improvements and advantages:

1. In the present disclosure, Sophora flavescens and Fructus cnidii in the external drug have the functions of clearing heat and dampness, dispelling wind, killing insects and relieving itching; through scientific extraction, the Sophora flavescens and Fructus cnidii combined with adjuvant medicinal materials such as Phellodendron amurense, Rhizoma atractylodis and Capillary artemisia are prepared into an effervescent suppository which can be directly applied to an inflammatory site so as to result in high local drug concentration, fast effect and even drug release with foams, thereby increasing the contact of the drug with the vaginal and cervical mucosa so that the drug can penetrate into the deep folds of the mucosa, thereby giving full play to the therapeutic effect of the drug.

2. In the present disclosure, the internal drug not only can reduce systemic use but also reduces adverse reactions caused by medication; the drug contains estradiol valerate, which is a precursor of the natural estrogen 17B-estradiol in a human body. During the use of estradiol valerate tablets, ovulation is not inhibited and endogenous hormone production is not significantly affected. Hormone replacement therapy can alleviate many symptoms of estrogen deficiency in postmenopausal women. Using an appropriate amount of estrogen in hormone replacement therapy (HRT) can reduce bone resorption, delay or prevent postmenopausal bone loss. When HRT is discontinued, the rate of bone loss is similar to that of postmenopausal women. There is no evidence to suggest that HRT restores bone mass to premenopausal levels. HRT also has a positive effect on skin collagen content and skin thickness, and can delay the rate of skin wrinkling.

3. In the present disclosure, the ingredients of the external drug are traditional Chinese medicines, which can have a neutralizing effect, in such the way, the use of the external drug while taking the internal drug can alleviate the side effects of the internal drug, the ovaries of women can be protected by combining with the effect of the internal drug, in such the way, the mutual complementation of the external drug and the internal drug maximizes the effect of the drug.

### BRIEF DESCRIPTION OF THE DRAWINGS

Next, the present disclosure will be further explained in combination with drawings and embodiments.

FIG. 1 is a diagram of ingredients tartaric acid, potassium hydrogen tartrate, boric acid and sodium bicarbonate of the present disclosure;

FIG. 2 is a diagram showing a foaming volume and orthogonal test results of a tablet of the present disclosure;

FIG. 3 is a diagram showing that a tablet of the present disclosure is affected by various factors;

FIG. 4 is a diagram showing a foaming volume and resolving time of the present disclosure; and

FIG. 5 is a diagram showing a drug content of a tablet released over time of the present disclosure over time.

### DETAILED DESCRIPTION OF THE EMBODIMENTS

Next, the present disclosure will be described in detail, the technical solutions in embodiments of the present disclosure will be clearly and completely described, obviously, the described embodiments are only some embodiments of the present disclosure but not all the embodiments. Based on the embodiments of the present disclosure, other embodiments obtained by persons of ordinary skill in the art without creative efforts are all included with the scope of protection of the present disclosure.

Through improvement, the present disclosure provides a method for extraction and preparation of an anti-inflammatory ovary-protecting herb. The technical solution of the present disclosure is as follows:

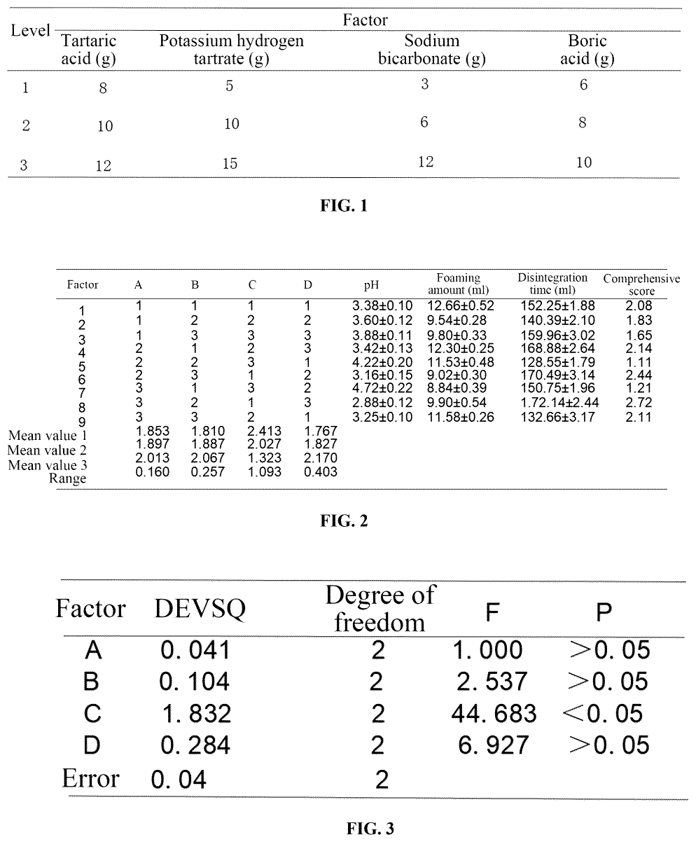

As shown in FIG. 1-FIG. 5, provided is a method for extraction and preparation of an anti-inflammatory ovary-protecting herb, comprising an internal drug and an external drug;
the external drug comprises 86 g of Sophora flavescens, 182 g of Fructus cnidii, 66 g of Phellodendron amurense, 146 g of Rhizoma atractylodis, 76 g of Capillary artemisia, matrix 5-40, sodium dihydrogen phosphate, and polysorbitol-80;
a decoction method is used to prepare the external drug, which comprises the following steps:

Step S1: weighing 86 g of Sophora flavescens, 182 g of Fructus cnidii, 66 g of Phellodendron amurense, 146 g of Rhizoma atractylodis and 76 g of Capillary artemisia, washing the above five medicine herbs and then placing the medicinal materials in a decoction pot, adding water above the surfaces of the medicinal materials to soak the medicinal materials for 30 min, decocting twice in total based on the decoction method, and then filtering using a filter screen to obtain filtrate; wherein the treatment and decoction method of the medicinal materials is as follows:
(1) removing impurities in Sophora flavescens, fructus cnidii, phellodendron amurense, Rhizoma atractylodis and Capillary artemisia, removing residual stems, soaking the above medicinal materials with water, fishing out the above medicinal materials, moisturizing, slicing, and drying for later use;
(2) cleaning up all the medicinal materials and then pouring the cleaned medicinal materials into a medicinal material tank, followed by pouring water into the medicinal material tank for decoction;
(3) putting 10 times the amount of water for the first decoction so that water covers the medicinal materials, and then setting decoction time as an hour and a half,
(4) collecting the liquid medicine herb obtained after the first decoction, putting 8 times the amount of water for the second decoction, and then setting decoction time as an hour and a half; and
(5) filtering two liquid medicinal materials, and then respectively placing two filtrates in different vessels;

Step S2: merging the two filtrates, performing vacuum concentration to 1000 ml, adding 3 times the amount of 95% ethanol after cooling, stirring while adding so that the content of alcohol in the merged filtrate reaches about 70%, placing for 24 h, filtering, collecting filtrate, recovering ethanol from the filtrate to form a solution, and then evaporating the solution until an extract is formed for later use; wherein the method for preparing the solution into the extract is as follows:
(1) shaking the collected solution, then putting the solution into a beaker and heating the solution, wherein in the process of heating, the temperature is raised so that ethanol is evaporated out; and
(2) adjusting the temperature to soft fire so that the solution is decocted to form the extract, and the density of the extract is 1.41-1.44 g/cm³;

Step S3: heating and melting the matrix S-40 in a water bath, adding the above extract, sodium dihydrogen phosphate and polysorbitol ester-80 under the condition of stirring, evenly mixing the above materials, pouring the obtained mixture into a bolt mould, cooling, and demoulding to obtain an ointment; wherein the preparation and formation method of the ointment is as follows:
(1) putting the extract, then evenly mixing sodium dihydrogen phosphate and polysorbitol ester-80, then pouring the obtained mixture into the extract, and subsequently adjusting the fire power to soft fire in the process of stirring;
(2) observing the viscosity of the ointment in the process of stirring until the ointment juice is not broken after being pulled, or the ointment juice is dropped into clean water to form beads without dispersing;
(3) putting the cooled ointment into a clean mould, covering the mouth of the mould with clean gauze before being not covered with a lid, placing the ointment overnight until complete cooling, and then taking out the ointment, that is, obtaining the ointment;
the internal drug comprises estradiol, sodium dodecyl sulfate, cross-linked polyvinylpyrrolidone, polyvinylpyrrolidone, cetanol, tartaric acid, potassium hydrogen tartrate, boric acid, sodium bicarbonate, magnesium stearate and sodium carboxymethyl cellulose; (Comprehensive score=|pH−5.0|+1/foaming amount+disintegration time/maximum disintegration time) is used as a measurement indicator to optimize the drug dosage. The lower the comprehensive score, the better the quality of the tablets. The various factors and levels are shown in FIG. 1, the experimental results are shown in FIG. 2, and the analysis of variance is shown in FIG. 3. It can be seen that the optimal composition of the effervescent agent is A1B1C3D1, i.e., 8 g of tartaric acid, 5 g of potassium hydrogen tartrate, 12 g of sodium bicarbonate, and 6 g of boric acid;
the specific steps for preparing tablets using an acid and alkali separated granulation method are as follows:

Step S4: screening estradiol via a 120-mesh sieve, and screening sodium carboxymethyl cellulose via a 80-mesh sieve for later use; wherein the method for grinding into powders is as follows:
(1) taking out the estradiol and removing a sugar coating outside the estradiol, and then grinding the estradiol into powders, wherein during the grinding, the ground powders are required to pass through a sieve with a size of 0.125 mm;
(2) grinding the sodium carboxymethyl cellulose into powders capable of passing through a sieve with a size of 0.180 mm, and respectively putting the above two drugs into different vessels;

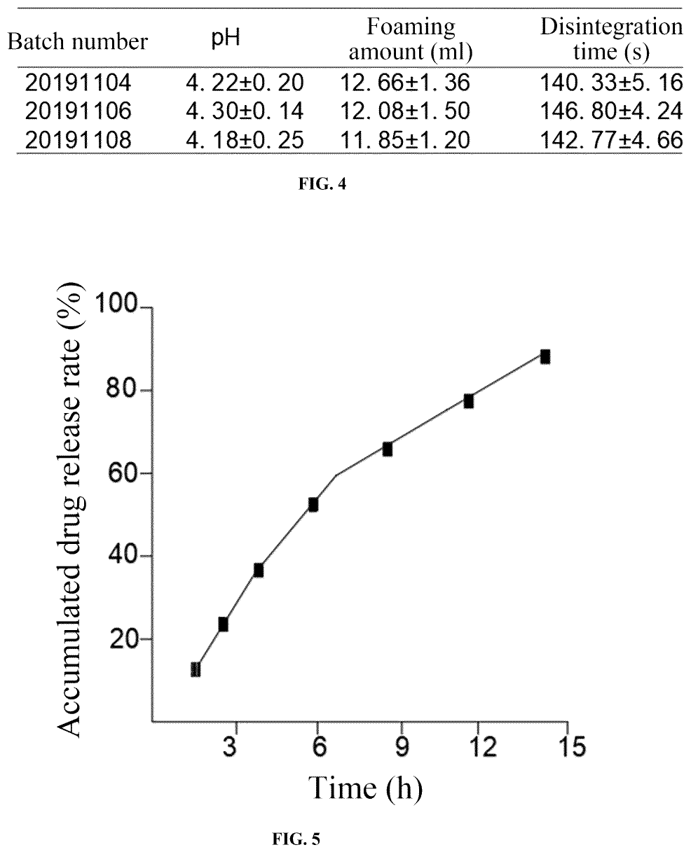

6 pieces of estradiol tablets and 6 pieces of blank tablets were taken, ground finely, 50 ml of water was added in the ground tablets so that the tablets produced a gas and foams, the tablets were fully dissolved and subjected to standing, and then the supernatant was taken to measure the pH. The average pH result is shown in Table 4 from which it can be seen that the aqueous solution of estradiol vaginal tablets is weakly acidic, close to the normal vaginal pH (3.8−4.4), and there is no significant difference compared to the average pH of blank tablets (4.22±0.26), indicating that estradiol does not affect the pH of the aqueous solution of the tablets. The foaming amount test shows that the foaming amount of each estradiol tablet exceeds 10 ml. The average foaming amount result is shown in FIG. 4, indicating that the foaming amount of estradiol vaginal tablets meets relevant regulations.

The preparation process for separated granulation using acid and alkali and then mixing and pressing tablets is adopted. In the prescription design, the dosage and variety of acidic substances were appropriately increased to make the pH of estradiol vaginal tablets weakly acidic after dissolution, in order to adapt to the normal physiological pH environment of the vagina and avoid significant irritation.

Step S5: evenly mixing the estradiol, boric acid, potassium hydrogen tartrate and tartaric acid, then adding the sodium carboxymethyl cellulose, 10% polyvinylpyrrolidone, and an anhydrous ethanol solution into the above mixture to prepare a soft material, and granulating after passing through a 20-mesh sieve; and drying wet particles in a thermostatic blower to obtain particles A; wherein the drying method of the particles A is as follows:
(1) mixing several materials and then adding the 10% polyvinylpyrrolidone and anhydrous ethanol into the above mixture;
(2) then twisting the obtained mixture into the particles A capable of passing through holes with a size of 1.27 mm during the stirring; and
(3) placing the twisted particles A in a drier, wherein each twisted particle is placed properly, and a distance between every two particles is equal;

Step S6: evenly mixing sodium bicarbonate, cross-linked polyvinylpyrrolidone, sodium dodecyl sulfate and cetanol, and then granulating using the same method, drying, and granulating; wherein the obtained particles are particles B, evenly mixing the two particles, then adding magnesium stearate into the above mixture to be mixed evenly, then measuring the content of the estradiol followed by determining the weight of the tablet, and tabletting; wherein the method for preparing the particles AB into the tablet is as follows:
(1) mixing and stirring the sodium bicarbonate, cross-linked polyvinylpyrrolidone, sodium dodecyl sulfate and cetanol, and observing the stirring;
(2) adding the 10% polyvinylpyrrolidone and anhydrous ethanol into the stirred liquid to be mixed, then twisting the particles B into small particles capable of passing through a sieve with a size of 1.27 mm, and then drying the particles B; and
(3) stirring the dried particles AB while putting magnesium stearate, then weighing the mixed particles AB on an electronic scale, and preparing the mixed particles into complete particles using a stamping tablet press when the weight of the mixed particles ALB meets the standard.

The experimental environment temperature is controlled as (37±0.5°) C, and the relative humidity is controlled below (75±5)%. Eight 20 ml test tubes were taken, 3 ml of 7% phosphate buffer (pH 4.5) was added into each tube, the tube wall was rotated to be moistened, the excess solution was poured out, 8 pieces of estradiol vaginal effervescent tablets were taken quickly and added into each of the above 8 test tubes, the test tubes were horizontally placed on a flat platform and slowly rolled, the slow effervescence of the preparation was observed, and the drugs on the test tubes were transferred to a 100 ml volumetric flask at 1, 2, 4, 6, 8, 10, 12, and 15 min after placement. Acetonitrile-methanol-water (60:10:30) is added for dilution until scale is reached, and then content of the estradiol is subjected to sampling inspection after shaking well and filtering. The results are shown in FIG. 5.

Further, in the step S1, the mesh size of the filter screen during the filtration is 0.150 mm, and the material of the filter screen is a nylon material. By designing the filter screen with a size of 0.150 mm, drug residues can be filtered out. By utilizing the characteristics of the filter screen made of nylon, such as wear resistance, high strength, acid alkali and corrosion resistance, low resistance and repeated washing, the filter screen can provide an effective filtration effect under multiple environments.

Further, in the step S2, an instrument for vacuum concentration is a rotary evaporator, and the vacuum degree in the instrument is 560 mmHg, arrangement of the rotary evaporator facilitates the treatment of those thermally sensitive or easily oxidizable compounds, because the boiling point of the liquid is relatively low in relatively low vacuum degree, so as to reduce the risk of thermal decomposition and oxidization, and utilization of a vacuum degree of 560 mmHg can help improve the distillation efficiency of a high-boiling-point or low-volatile compound.

Further, in the step S2, the recovery of ethanol from the filtrate adopts a distillation method, and the heating temperature of the filtrate is 78-90° C. The impurities in the filtrate can be effectively removed by using a distillation method to obtain ethanol with relatively high purity. By setting the temperature as 78-90° C., ethanol starts boiling and is transformed into a gas state, that is to say, it can be gradually heated and evaporated until all of the ethanol is transformed into the gas state, which is beneficial for recovery.

Further, in the step S3, the mould is cylindrical, and the height of the mould is 0.2 m. by setting the shape of the mould, the storage of the ointment is facilitated. By utilizing the height of the mould, the ointment is convenient to use.

Further, in the step S4, an instrument for grinding is a mortar, and the material of the mortar is porcelain. By arranging the mortar, the solid matters are convenient to grind, so as to facilitate the mixing of the powdered solids. The mortar is made of porcelain, and therefore it has the characteristics of hardness and wear resistance, which makes it bear the repeated grinding of a pestle and allows the mortar not to be easily abraded, thereby ensuring the accuracy and reliability of the experimental results.

Further, in the step S5, the time of wet mixing and stirring is 2-3 min, and the drying temperature is 35-45° C. By setting the stirring time as 2-3 min, it is ensured that the materials can be sufficiently and evenly mixed so as to avoid too high or too low local concentration. In the process of wet mixing, the materials are mixed with water or other liquids to form certain humidity so that the materials are more easily and evenly stirred. By utilizing the drying temperature of 35-45° C., it is avoided that the materials are denatured or damaged in the process of drying, a lower drying temperature can ensure the gradual evaporation of water in the materials but does not cause the change in structure or property of materials.

Further, in the step S5, a device for twisting into particles is an oscillating granulator, and the material of the sieve is stainless steel. Because the filter screen made of stainless steel used in the arranged oscillating granulator has the advantages of corrosion resistance, high temperature resistance, good screening effect and the like, and is easy to clean and maintain, the properties and service life of the device can be improved, so as to bring better benefits for production.

Further, in the step S6, the model of the stamping tablet press is a dual-discharge tablet press, and the weight of the tablet is 1 g. By arranging the dual-discharge tablet press, the particles can receive more even force distribution in the tablet press, thereby ensuring the consistency and stability of the tablet quality. In addition, the stamping tablet press has strong tabletting force, effectively ensuring the formation quality of different drugs so as to achieve the stable and reliable effect. The tablet is convenient to take due to its weight.

The above description makes those skilled in the art achieve or use the preset disclosure. Various modifications to these embodiments will be apparent to professionals in the art, and the general principles defined herein can be implemented in other embodiments without departing from the spirit or scope of the present disclosure. Therefore, the present disclosure will not be limited to the embodiments shown herein, but will conform to the widest range consistent with the principles and novel features disclosed herein.

What is claimed is:

1. A method for extraction and preparation of an anti-inflammatory ovary-protecting herb, comprising an internal drug and an external drug,
wherein the external drug comprises 86 g of Sophora flavescens, 182 g of Fructus cnidii, 66 g of Phellodendron amurense, 146 g of Rhizoma atractylodis, 76 g of Capillary artemisia, matrix S-40, sodium dihydrogen phosphate, and polysorbitol-80;
a decoction method is used to prepare the external drug, which comprises the following steps:
Step S1: weighing 86 g of Sophora flavescens, 182 g of Fructus cnidii, 66 g of phellodendron amurense, 146 g of Rhizoma atractylodis and 76 g of Capillary artemisia, washing the above five medicine herbs and then placing the medicinal materials in a decoction pot, adding water above the surfaces of the medicinal materials to soak the medicinal materials for 30 min, decocting twice in total based on the decoction method, and then filtering using a filter screen to obtain filtrate; wherein the treatment and decoction method of the medicinal materials is as follows:
(1) removing impurities in Sophora flavescens, fructus cnidii, Phellodendron amurense, Rhizoma atractylodis and Capillary artemisia, removing residual stems, soaking the above medicinal materials with water, followed by fishing out, moisturizing and slicing the above medicinal materials in sequence, and drying for later use;
(2) cleaning up all the medicinal materials and then pouring the cleaned medicinal materials into a medicinal material tank, followed by pouring water into the medicinal material tank for decoction;
(3) putting 10 times the amount of water for the first decoction so that water covers the medicinal materials, and then setting decoction time as an hour and a half,
(4) collecting the liquid medicine herb obtained after the first decoction, putting 8 times the amount of water for the second decoction, and then setting decoction time as an hour and a half, and
(5) filtering two liquid medicinal materials, and then respectively placing two filtrates in different vessels;
Step S2: merging the two filtrates, performing vacuum concentration to 1000 ml, adding 3 times the amount of 95% ethanol after cooling while stirring so that the content of alcohol in the merged filtrate reaches about 70%, placing for 24 h, filtering, collecting filtrate, recovering ethanol from the filtrate to form a solution, and then evaporating the solution until an extract is formed for later use; wherein the method for preparing the solution into the extract is as follows:
(1) shaking the collected solution, then putting the solution into a beaker and heating the solution, wherein in the process of heating, the temperature is raised so that ethanol is evaporated out; and
(2) adjusting the temperature to soft fire so that the solution is decocted to form the extract, and the density of the extract is 1.41-1.44 g/cm³;
Step S3: heating and melting the matrix S-40 in a water bath, adding the above extract, sodium dihydrogen phosphate and polysorbitol ester-80 under the condition of stirring, evenly mixing the above materials, pouring the obtained mixture into a bolt mould, cooling, and demoulding to obtain an ointment; wherein the preparation and formation method of the ointment is as follows:
(1) putting the extract, then evenly mixing sodium dihydrogen phosphate and polysorbitol ester-80, then pouring the obtained mixture into the extract, and subsequently adjusting the fire power to soft fire in the process of stirring;
(2) observing the viscosity of the ointment in the process of stirring until the ointment juice is not broken after being pulled, or the ointment juice is dropped into clean water to form beads without dispersing; and
(3) putting the cooled ointment into a clean mould, covering the mouth of the mould with clean gauze before being not covered with a lid, placing the ointment overnight until complete cooling, and then taking out the ointment;
the internal drug comprises estradiol, sodium dodecyl sulfate, cross-linked polyvinylpyrrolidone, polyvinylpyrrolidone, cetanol, tartaric acid, potassium hydrogen tartrate, boric acid, sodium bicarbonate, magnesium stearate and sodium carboxymethyl cellulose;
the specific steps for preparing tablets using an acid and alkali separated granulation method are as follows:
Step S4: screening estradiol via a 120-mesh sieve, and screening sodium carboxymethyl cellulose via a 80-mesh sieve for later use; wherein the method for grinding into powders is as follows:
(1) taking out the estradiol and removing a sugar coating outside the estradiol, and then grinding the estradiol into powders, wherein during the grinding, the ground powders are required to pass through a sieve with a size of 0.125 mm; and
(2) grinding the sodium carboxymethyl cellulose into powders capable of passing through a sieve with a size of 0.180 mm, and respectively putting the above two drugs into different vessels;
Step S5: evenly mixing the estradiol, boric acid, potassium hydrogen tartrate and tartaric acid, then adding the sodium carboxymethyl cellulose, 10% polyvinylpyrrolidone, and an anhydrous ethanol solution into the above mixture to prepare a soft material, and granulating after passing through a 20-mesh sieve; and drying wet particles in a thermostatic blower to obtain particles A; wherein the drying method of the particles A is as follows:
(1) mixing several materials and then adding the 10% polyvinylpyrrolidone and anhydrous ethanol into the above mixture;
(2) then twisting the obtained mixture into the particles A capable of passing through holes with a size of 1.27 mm during the stirring; and
(3) placing the twisted particles Ain a drier, wherein each twisted particle is placed properly, and a distance between every two particles is equal;
Step S6: evenly mixing sodium bicarbonate, cross-linked polyvinylpyrrolidone, sodium dodecyl sulfate and cetanol, and then granulating using the same method, drying, and granulating;
wherein the obtained particles are particles B, evenly mixing the two particles, then adding magnesium stearate into the above mixture to be mixed evenly, then measuring the content of the estradiol followed by determining the weight of the tablet, and tabletting; wherein the method for preparing the particles AB into the tablet is as follows:
(1) mixing and stirring the sodium bicarbonate, cross-linked polyvinylpyrrolidone, sodium dodecyl sulfate and cetanol, and observing the stirring;
(2) adding the 10% polyvinylpyrrolidone and anhydrous ethanol into the stirred liquid to be mixed, then twisting the particles B into small particles capable of passing through a sieve with a size of 1.27 mm, and then drying the particles B; and
(3) stirring the dried particles AB while putting magnesium stearate, then weighing the mixed particles AB on an electronic scale, and preparing the mixed particles into complete particles using a stamping tablet press when the weight of the mixed particles AB meets the standard.

2. The method for extraction and preparation of the anti-inflammatory ovary-protecting herb according to claim 1, wherein in the step S1, the mesh size of the filter screen during the filtration is 0.150 mm, and the material of the filter screen is a nylon material.

3. The method for extraction and preparation of the anti-inflammatory ovary-protecting herb according to claim 1, wherein in the step S2, an instrument for vacuum concentration is a rotary evaporator, and the vacuum degree in the instrument is 560 mmHg.

4. The method for extraction and preparation of the anti-inflammatory ovary-protecting herb according to claim 1, wherein in the step S2, the recovery of ethanol from the filtrate adopts a distillation method, and the heating temperature of the filtrate is 78-90° C.

5. The method for extraction and preparation of the anti-inflammatory ovary-protecting herb according to claim 1, wherein in the step S3, the mould is cylindrical, and the height of the mould is 0.2 m.

6. The method for extraction and preparation of the anti-inflammatory ovary-protecting herb according to claim 1, wherein in the step S4, an instrument for grinding is a mortar, and the material of the mortar is porcelain.

7. The method for extraction and preparation of the anti-inflammatory ovary-protecting herb according to claim 1, wherein in the step S5, the time of wet mixing and stirring is 2-3 min, and the drying temperature is 35-45° C.

8. The method for extraction and preparation of the anti-inflammatory ovary-protecting herb according to claim 1, wherein in the step S5, a device for twisting into particles is an oscillating granulator, and the material of the sieve is stainless steel.

9. The method for extraction and preparation of the anti-inflammatory ovary-protecting herb according to claim 1, wherein in the step S6, the model of the stamping tablet press is a dual-discharge tablet press, and the weight of the tablet is 1 g.
